(19) United States
(12) Patent Application Publication
(10) Pub. No.: US 2004/0015097 A1
(43) Pub. Date: Jan. 22, 2004

(54) Method and device for diagnostically determining the receptivity of the uterine epithelium

(76) Inventors: Hans-Werner Denker, Essen (DE); Michael Thie, Essen (DE); Hanna Tinel, Dortmund (DE)

Correspondence Address: PROCOPIO, CORY, HARGREAVES & SAVITCH LLP, 530 B STREET, SUITE 2100, SAN DIEGO, CA, 92101, US

(21) Appl. No.: 10/239,949

(22) PCT Filed: Mar. 20, 2001
(86) PCT No.: PCT/DE01/01061

(30) Foreign Application Priority Data
Mar. 24, 2000 (DE) 10014350.4

(51) Int. Cl. A61B 10/00

(52) U.S. Cl. 600/551

(57) ABSTRACT

The present invention relates to a method for diagnostic determination of the receptivity of the uterine epithelium, in particular the time of the window of implantation within the menstrual cycle, and to a device for carrying out the method.

The present invention relates to a method for diagnostic determination of the receptivity of the uterine epithelium, in particular the time of the window of implantation within the menstrual cycle, and to a device for carrying out the method.

In the treatment of human infertility, increasing importance has in recent years been attached to methods of artificial fertilization, in particular to what is called in vitro fertilization, and intracytoplasmatic sperm injection. In these treatment methods, egg cells are fertilized outside the uterus and are then transferred at an embryonic stage into the uterus.

It has long been recognized, with regard to embryo implantation, that the attachment of trophoblast cells to uterine epithelial cells in the initial phase is possible only when all the constituents of the endometrium, that is to say including the uterine epithelium, have attained a certain physiological state. The receptivity of the uterus determines the window of implantation within the menstrual cycle and is regulated by the secretion of oestrogen and progesterone by the ovary. The receptive phase in humans begins about 4 to 5 days after fertilization and continues for about 4 to 8 days.

The exact course of events in the initial processes of attachment at the start of implantation in humans is not yet fully understood. In all attempts to treat infertility, it is this phase which still causes the greatest difficulties, because the sequence of events and the regulation of these processes are largely unknown.

The pregnancy success rates in in vitro fertilization and embryo transfer therapy (IVF/ET therapy) are only between 10% and 20% worldwide in relation to the embryo transfers performed. A specific medical treatment of defects of implantation is not yet possible. The only available way of supporting the periimplantation phase is through endocrinology, for example by means of steroid replacement (also indirectly using hCG for example).

The implantation of the embryo is of central importance for the development of a pregnancy and is initiated by a direct interaction of the trophoblast with the luminal epithelium of the uterus. Because of the relative inaccessibility of the procedure, only a small amount of reliable information is available concerning the cell biology processes which underlie the implantation of the human embryo. An understanding of these underlying cellbiology processes would however be desirable not only for an appreciation of the cell-to-cell adhesion between the blastocyst and the endometrial mucosa, which adhesion is so critical for embryo implantation, but would also be of considerable interest for a more effective hormone treatment of fertility problems of endometrial origin.

On the basis of experimental trials conducted by the inventors, observations have been made and intellectual concepts developed which make it possible to effectively analyze the suspected cell biology principles involved.

The inventors discovered that the formation of a structurally and functionally specialized cell-cell contact between epithelial cells of the trophoblast of the blastocyst ready for implantation and epithelial cells on the surface of the receptive endometrium is of particular importance for embryo implantation.

In doing so, the inventors found that the physicochemical and biochemical processes surrounding the recognition, convergence and binding between the apical (free) plasma membrane domain of the cells of trophoblast and uterine epithelium should not be seen as an isolated membrane phenomenon, but as a complex of many reaction steps which are controlled within these cells.

In the context of said trials, the inventors found that, upon cell contact between trophoblast and uterine epithelium, a change is detected in the intracellular calcium ion concentration in the contact area and in the environment surrounding the latter. Upon stimulation of the cells of the uterine epithelium, a temporary increase was detected in the intracellular free calcium in the uterine epithelium.

In this connection, however, it was found that a stimulation of the epithelial cells led to an increase in intracellular calcium only in those cells which have a heightened degree of adhesion with respect to the trophoblast and are thus in a state of heightened receptivity.

As regards its kinetics and its spatial propagation, this increase in calcium has characteristics which can be made use of when assessing the state of receptivity.

In light of these findings, the inventors have developed a method for determining heightened receptivity of the uterine epithelium, which method is based on the detection of a change in the intracellular ion concentration and/or ion distribution, in particular of the calcium ions, and, according to a subsidiary aspect of the present invention, on the associated change in the transepithelial electrical resistance.

The present invention is therefore directed at a method for diagnostic determination of the receptivity of the uterine epithelium, comprising the following steps:

a) determining the initial value of the ion concentration of the uterine epithelium, or its transepithelial resistance, and storing the results obtained;

b) stimulating the uterine epithelium physically and/or chemically; and

c) determining the change which the stimulation has brought about in the value of the ion concentration of the uterine epithelium, or its transepithelial resistance, and evaluating the result obtained, taking into consideration the value which was obtained in step a).

Physical or chemical stimulation is to be understood as stimulation of the epithelium by means of an electrical voltage, preferably via an electrode located on the probe, as will be discussed by way of example later, and by means of which the resistance measurement can also be carried out, or by mechanical stimulation by exerting pressure, or by application of a chemical substance.

In this method, the changes which the stimulation has brought about in the ion concentration are preferably determined by fluorescence optical measurement or by measurement using an ion-sensitive biosensor and/or ion sensor, or changes in the transepithelial electrical resistance are determined, and, in step c), the determination of these parameters preferably takes into consideration the kinetic profile of the change in the measured parameter.

The determination of the ion concentration moreover preferably includes the kinetic profile of the measured parameters inside and/or outside the area of the directly stimulated cells.

It will be obvious to the skilled person that it is not generally necessary to know the absolute values of the ion concentration in order to assess the state of receptivity of the uterine mucosa or to fine-tune hormone treatment. Instead, for these purposes, it is generally enough that stimulation of the uterine mucosa results in a change in the intracellular calcium ion concentration, in the passage of ions through the cell membrane or in the transepithelial resistance. In one embodiment of the method according to the invention, a change in the transepithelial electrical resistance serves as an indicator of a change in the intracellular ion concentration, in particular the calcium ion concentration, without its being necessary for this purpose, for example, to know the absolute ion concentrations or calcium ion concentrations before and after the stimulation.

As secondary messengers, calcium ions bring about a large number of subsequent intracellular reactions which, by what are for the most part hitherto unknown mechanisms, also cause a change in the transmembranous ion flows, regulated by various ion channels, and thus in the measured conductivity or the transepithelial resistance. Consequently, awareness of a change in the transepithelial conductivity permits a conclusion to be drawn regarding the state of receptivity of the uterine mucosa. The increased intracellular calcium ion concentration resulting from the stimulation of the uterine epithelium is one of the causes of the change in the transepithelial resistance. By monitoring this parameter, before and after stimulation, it is thus also possible to form an opinion regarding the receptivity of the uterine epithelium at that moment.

In a preferred embodiment of the method according to the invention, a measurement of the transepithelial electrical resistance is carried out, involving, in succession, the determination of the basic value of the transepithelial resistance, stimulation, and the determination of the changed value. The increased intracellular ion concentration produced by the stimulation of the uterine epithelium results, through subsequent reactions, in a change in the membrane conductivity and thus also results in a change in the transepithelial resistance. Since the ion shifts caused by the stimulation also correlate with the changes in the measured electrical resistance, the determination of the electrical resistance on the uterine mucosa can be used as a measure of the reactivity.

Moreover, the inventors propose a further embodiment of the method of the present invention, which further embodiment is directed at a method for diagnostic determination of the receptivity of the uterine epithelium, said method comprising the following steps:

a) determining the initial value of the calcium ion concentration of the uterine epithelium, and storing the result obtained;

b) stimulating the uterine epithelium physically and/or chemically; and

c) again determining the calcium ion concentration of the uterine epithelium after the stimulation, and evaluating the result obtained, taking into consideration the value which was obtained in step a).

In another method based on the latter measurement of the calcium ion concentration, preferably by fluorescence optical measurement, the method comprises steps a) to f):

a) applying a calcium indicator to the uterine epithelium, which calcium indicator can be a fluorescent/luminescent indicator;

b) irradiating the uterine epithelium with light of suitable wavelength, if fluorescent indicators are used, and measuring the radiation (fluorescent/luminescent radiation) emitted by the calcium indicator;

c) evaluating the resulting measurement values in an evaluation unit;

d) stimulating the uterine epithelium, if appropriate, physically and/or chemically;

e) once again irradiating the uterine epithelium with light of suitable wavelength, if fluorescent indicators are used, and measuring the radiation (fluorescent and/or luminescent radiation) emitted by the calcium indicator; and

f) evaluating the resulting measurement values in an evaluation unit.

In a further method, in contrast to the fluorescence optical determination of the calcium ion concentration, the latter is determined by means of a biosensor or ion sensor which is sensitive to calcium ions. The technical principles of these detection methods are well known in the prior art, so that it is possible for the skilled person to select suitable bioaffinity sensors, biomimetric sensors or calcium ion sensors for the method according to the invention.

By means of the method according to the invention, it is possible to determine the time of heightened receptivity and thus increase the chances of successful union between trophoblast and uterine epithelium and, in the final analysis, to increase the chance of successful implantation of the embryo. Moreover, this method can be advantageously used to fine-tune the hormonal treatment of fertility problems of endometrial origin.

As an advantageous embodiment of the method according to the invention, the inventors propose that the method according to the invention be performed in analogy to a hysteroscopy and that a probe or an endoscope be correspondingly introduced into the uterine cavity. The uterine cavity may optionally be opened out with a physiological saline solution which contains calcium ions, in order to irrigate the surface in preparation or to inspect it.

To carry out an embodiment of the method according to the invention, the inventors have proposed a diagnostic device which comprises, as its main components, a specially designed probe and an evaluation unit attached to the latter.

In one embodiment, the diagnostic device for carrying out the method according to the invention comprises, in one variant, a probe which, in its simplest version, can consist of a probe rod or tube or a flexible tube, if appropriate equipped with an optical system for observing the uterine mucosa, and also an evaluation unit which is functionally connected to the probe and is used to evaluate the measurement values obtained, said probe further comprising:

a) means for stimulating the uterine epithelium physically and/or chemically,

b) a measurement head, located preferably at the uterine end of the probe, with means for determining the epithelial ion concentration or the transepithelial resistance.

In a preferred embodiment, the diagnostic device for carrying out the method has an optical system for observing the uterine mucosa, which optical system is integrated in the probe rod or tube for introduction into the uterus.

In a further preferred embodiment, the measurement head has at least one electrode for measuring the transepithelial electrical resistance. In addition, means are preferably provided for sealing off the at least one electrode from its environment.

In a further preferred embodiment, the diagnostic device has a further electrode arranged as a neutral electrode outside or inside the uterus.

Preferred diagnostic devices have a measurement head for determining the ion concentration, which measurement head comprises means for fluorescence optical measurement and/or an ion-sensitive biosensor and/or ion sensor.

For the fluorescence optical method, a calcium indicator and, if appropriate, further test substances can be applied to the uterine epithelium via the preferably multi-lumen probe for introduction into the uterus. This can be done, for example, via a channel in the probe tube. For this purpose, a storage vessel for the solution containing the calcium indicator can also be connected to the probe, or can be provided on the latter, from which storage vessel this solution is conveyed through a delivery line by means of a delivery device, for example a pump, to the application opening near the measurement head. To limit the amount of solution applied, a dosing device is preferably provided which is designed in the manner of a syringe.

A preferred diagnostic device for determining the epithelial calcium ion concentration has a measurement head which is designed for radiating light of suitable wavelength and for measuring emitted light.

In cases where the introduction of an exogenous fluorescence indicator is not necessary, since endogenous fluorescent compounds which may also be present are able to indicate the change in the calcium concentration, the application of an “exogenous” calcium indicator and thus the aforementioned means for its application are not required.

The probe for fluorescence optical measurement is provided with a measurement head through which light of suitable wavelength is radiated for fluorescence excitation, and emitted light is received by a photoelement integrated in the measurement head.

Means can also be provided in the probe for the purpose of introducing additional devices which permit and control manipulations even with the measurement head in the lumen of the uterus. Such devices can, for example, be structural parts which permit a mechanical advance of the measurement head itself, or a mechanical stimulator controlled by a micro-manipulator.

In addition, the probe rod or tube can also have an optical channel, such as a fibre optic, and the measurement head can contain a corresponding lens for inspecting the surface of the mucosa.

Instead of a measurement head with an integrated light source and photoelement, a light source which radiates light of suitable wavelength for excitation of the fluorescence indicator, and which is connected to the probe via a fibre optic guide so that the emitted radiation can be delivered to the uterine epithelium, and a photoelement for detection of the fluorescence can be provided on or connected to that end of the probe tube which lies outside the body during the examination.

The measured values can be evaluated in the attached evaluation unit. The evaluation unit, for example a computer system with software which can be run on it, (a) permits the recording and storage of the detected signals and (b) can perform the evaluation of these signals.

The measurement values are evaluated by signal evaluation, based on the parameters of “size” and “propagation” of the calcium signal or the change in the transepithelial resistance. The size of the signal is obtained from the difference between the base value before stimulation and the value after stimulation. To increase the measurement accuracy, it is advantageous to use at least one further light inlet/outlet opening on the measurement head, or additional electrodes, in order to detect the spatial propagation of the signal and to take this into consideration in the evaluation.

If so required, the calcium indicator can preferably be applied in the form of an ester compound which spontaneously penetrates the cell membrane, is cleaved intracellularly by esterases, and then, in the cleaved form, can no longer leave the cell. Using other indicators, the transport into the cells can also be effected by electroporation, coupling to liposomes or ligands for cell-surface-resistant receptors with subsequent endocytosis, or by spontaneous uptake by the cells.

To record the base value when using the fluorescence principle, excitation of the intracellular indicator is effected using light of suitable wavelength, which is not necessary in the alternative luminescence measurement. The measurement can be carried out in vivo by integral measurement of the emitted radiation, for example fluorescent light, within a window of defined surface area.

After chemical or physical stimulation of the uterine epithelial cells, the measurement of the emitted radiation is repeated, and the difference between the signals from the measurement before stimulation and the measurement after stimulation, and the time-dependent kinetics, are recorded and evaluated in the evaluation unit.

The same applies to the method for measuring the transepithelial resistance, with the difference that in this case, instead of the optical system, electrodes are used to determine electrical parameters such as conductivity or electrical resistance.

The probe which can be used in the method according to the invention should comprise two independent measurement windows or electrodes of this kind, located at a defined distance from one another, so as to be able also to record the propagation of the signal in the surface. The head of the probe tube also comprises the openings (inflow and outflow) of channels for irrigation fluid and, if appropriate, a normal lens system of an endoscope (=hysteroscope) for viewing the surface of the mucosa.

When the method is applied in vivo, the probe tube is introduced into the uterine cavity and the measurement head is placed against the surface of the mucosa. If so required, the uterine cavity is flushed clear by irrigation with a calcium-containing physiological solution. Thereafter, when using the fluorescence optical method, the calcium indicator solution is introduced if necessary through the irrigation channel, with said calcium indicator solution being taken up by the epithelial cells analogously to the manner described above.

The measurement procedures for determining the starting value and for determining the value after stimulation are as described above. The stimulation of the uterine epithelium between the two measurement procedures can be done by touching it with the whole head of the tube or by using a device which is guided through a separate channel in the probe tube.

In a further preferred embodiment of a diagnostic device in which the epithelial ion concentration is measured by biosensor and/or ion sensor, a measurement head is provided which comprises at least one ion-sensitive, preferably calcium-ion-sensitive, biosensor and/or ion sensor.

Moreover, all of the proposed diagnostic devices preferably have means for introduction of additional devices which permit and control manipulations within the lumen of the uterus.

1. Method for diagnostic determination of the receptivity of the uterine epithelium, comprising the following steps:
a) determining the initial value of the ion concentration of the uterine epithelium, or its transepithelial resistance, and storing the results obtained;
b) stimulating the uterine epithelium physically and/or chemically; and
c) determining the change which the stimulation has brought about in the value of the ion concentration of the uterine epithelium, or its transepithelial resistance, and evaluating the result obtained, taking into consideration the value which was obtained in step a).

2. Method according to claim 1, in which the ion concentration is determined by fluorescence optical measurement, or by measurement using an ion-sensitive biosensor and/or ion sensor.

3. Method according to claim 2 in which, in step c), the determination of the parameters takes into consideration the kinetic profile of the concentration change or of the transepithelial resistance.

4. Method according to claim 3 wherein said ion is calcium and in which determining the calcium ion concentration, is carried out by fluorescence optical measurement, by measurement of the transepithelial electrical resistance, or by measurement using an ion-sensitive biosensor and/or ion sensor.

5. Method according to claim 1, in which the transepithelial electrical resistance is measured before and after the stimulation, which is preferably produced by an electrical stimulus of the epithelium, and the measurement values obtained are compared with one another.

6. Method according to claim 5 in which, in step c), the determination of the parameters takes into consideration the kinetic profile of the concentration change or of the transepithelial resistance.

7. Method according to claim 6 wherein said ion is calcium and in which determining the calcium ion concentration, is carried out by fluorescence optical measurement, by measurement of the transepithelial electrical resistance, or by measurement using an ion-sensitive biosensor and/or ion sensor.

8. Diagnostic device for a method of determining the receptivity of the uterine epithelium, said method comprising the following steps a) determining the initial value of the ion concentration of the uterine epithelium, or its transepithelial resistance, and storing the results obtained;
b) stimulating the uterine epithelium physically and/or chemically; and
c) determining the change which the stimulation has brought about in the value of the ion concentration of the uterine epithelium, or its transepithelial resistance, and evaluating the result obtained, taking into consideration the value which was obtained in step a), which device comprises a probe, if appropriate equipped with an optical system for observing the uterine mucosa, and also an evaluation unit which is functionally connected to the probe and is used to evaluate the measurement values obtained, said probe further comprising:
a) means for stimulating the uterine epithelium physically and/or chemically,
b) a measurement head, located preferably at the uterine end of the probe, with means for determining the epithelial ion concentration or the transepithelial resistance.

9. Diagnostic device according to claim 8 for carrying out the method, in which an optical system for observing the uterine mucosa is integrated in the probe for introduction into the uterus.

10. Diagnostic device according to claim 9 in which the measurement head for determining the epithelial ion concentration has at least one electrode for measuring the transepithelial electrical resistance.

11. Diagnostic device according to claim 10, which additionally has means for sealing off the at least one electrode from its environment.

12. Diagnostic device according to claim 11, which has a further electrode arranged as a neutral electrode outside or inside the uterus.

13. Diagnostic device according to claim 8 in which the measurement head for determining the ion concentration is designed with means for fluorescence optical measurement, or with at least one ion-sensitive biosensor and/or ion sensor.

14. Diagnostic device of claim 13 in which in order to determine the epithelial ion concentration by means of measurement by biosensor and/or ion sensor, the measurement head has at least one ion-sensitive biosensor and/or ion sensor.

15. Diagnostic device according to claim 13, in which the measurement head is designed for determining the calcium ion concentration by fluorescence optical measurement and, if appropriate, additionally comprises means for applying a calcium indicator and/or, if appropriate, further substances onto the uterine epithelium via the tube of the probe.

16. Diagnostic device according to claim 15, in which the measurement head for determining the epithelial calcium ion concentration by means of fluorescence optical measurement is designed as a measurement head for radiating light at a suitable wavelength and for measuring emitted light.

17. Diagnostic device according to claim 16 in which the measurement head had at least one biosensor and/or ion sensor sensitive to calcium ions.

18. Diagnostic device according to claim 8 which additionally comprises means for introduction of additional devices which permit and control manipulations within the lumen of the uterus.
